(19) United States
(12) Patent Application Publication
(10) Pub. No.: US 2016/0317682 A1
(43) Pub. Date: Nov. 3, 2016

(54) PREPARATION OF Ag18F AND ITS USE IN THE SYNTHESIS OF PET RADIOTRACERS

(71) Applicant: THE REGENTS OF THE UNIVERSITY OF MICHIGAN, Ann Arbor, MI (US)

(72) Inventors: Peter J. H. Scott, Ypsilanti, MI (US); Allen F. Brooks, Ann Arbor, MI (US); Naoko Ichiishi, Ann Arbor, MI (US); Melanie S. Sanford, Ann Arbor, MI (US)

(73) Assignee: THE REGENTS OFTHE UNIVERSITY OF MICHIGAN, Ann Arbor, MI (US)

(21) Appl. No.: 15/142,131

(22) Filed: Apr. 29, 2016

Related U.S. Application Data
(60) Provisional application No. 62/155,433, filed on Apr. 30, 2015.

(51) Int. Cl. A61K 51/04; C07C 227/16; C01G 5/00; C07C 17/20

(52) U.S. Cl. CPC A61K 51/04; C07C 17/208; C07C 227/16; C01G 5/003

(57) ABSTRACT

Copper-catalyzed radiofluorination of aryl iodides using Ag¹⁸F, methods of preparing Ag¹⁸F, and compounds obtained by copper-catalyzed radiofluorination of aryl iodides using Ag¹⁸F are disclosed. Diagnostic and therapeutic methods involving such compounds also are disclosed.

### CROSS-REFERENCE TO RELATED APPLICATIONS

This application claims the benefit of U.S. Provisional Patent Application No. 62/155,433, filed Apr. 30, 2015, which is incorporated by reference herein in its entirety.

### GOVERNMENT RIGHTS

This invention was made with government support under grant numbers GM073836 and T32-EB005172 awarded by the National Institute of Health. The government has certain rights in the invention.

### BACKGROUND

1. Field of the Invention

The present invention generally relates to methods of preparing Ag¹⁸F, to methods of radiofluorination using Ag¹⁸F, to compounds obtained by radiofluorination using Ag¹⁸F, and to diagnostic and therapeutic methods involving such compounds.

2. Brief Description of Related Technology

Positron emission tomography (PET) is a powerful and minimally invasive medical imaging technique that provides kinetic physiochemical information. The most commonly used radioisotope for PET is ¹⁸F, which offers the advantages of high resolution imaging (ca. 2.5 mm in tissue), a relatively long lifetime (t_{1/2}=110 min), and minimal perturbation of radioligand binding. Despite these advantages, the development of novel ¹⁸F radiotracers is currently impeded by a paucity of general and effective radiofluorination methods. There are currently few robust synthetic procedures for the incorporation of ¹⁸F into organic molecules with sufficient speed, selectivity, yield, radiochemical purity, and reproducibility to provide clinical imaging materials. Direct methods for the late stage nucleophilic [¹⁸F]fluorination of electron-rich aromatic substrates remains an especially long-standing challenge in the PET community. A target of particular interest in this regard is 6-[¹⁸F]fluoro-_{L}-DOPA (6-[¹⁸F]fluoro-_{L}-3,4-dihydroxyphenylalanine), which serves as a valuable diagnostic for probing the regional distribution of dopamine in the human brain. While there has been much activity in the radiofluorination community aimed at accessing 6-[¹⁸F]fluoro-_{L}-DOPA, current methods suffer from drawbacks (including low specific activity, multi-step procedures, chiral separations, and/or poor yield) that limit routine production of this material.

The majority of radiofluorination methods for electron rich aryl rings utilize electrophilic fluorinating reagents derived from [¹⁸F]F₂. However, [¹⁸F]F₂ production typically requires ¹⁹F₂ as a carrier gas, which leads to low specific activity (SA) radiotracers (typically <1.0 Ci/mmol) and requires specialized facilities. The development of [¹⁸F]KF production from [¹⁸O]water has provided the means to synthesize high SA radiotracers (>1,000 Ci/mmol) through nucleophilic substitution (typically S_{N}2 or S_{N}Ar). However, the use of [¹⁸F]KF is generally limited to the formation of primary sp³-C—F bonds or sp²-C—F bonds on activated electron deficient aromatics.

Two main strategies have been used to address these limitations. The first involves radiofluorination of powerful electrophiles such as diaryliodonium salts. Diaryliodonium salts bearing the 2-thienyl group have been shown to react with [¹⁸F]KF at elevated temperatures (often ≧150° C.) to afford [¹⁸F]fluororarenes (Scheme 1). In these systems, the 2-thienyl group directs radiofluorination to the other aromatic ligand on iodine, with moderate to good selectivity. However, the [(thienyl)(aryl)I⁺] starting materials are often challenging to prepare, suffer from low stability, and have a limited shelf-life. Furthermore, with electron neutral or rich substrates, these transformations frequently require high temperatures, exhibit modest regioselectivity, demonstrate limited functional group tolerance, and provide low radiochemical yields. As such, it has proven challenging to access important radiotracers, most notably 6-[¹⁸F]fluoro-_{L}-DOPA derivatives, using this method.

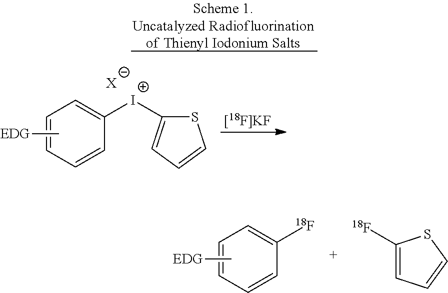

A second strategy applies transition metal catalysts and/or reagents to achieve nucleophilic radiofluorination. Transition metal catalysis offers opportunities for accelerating radiofluorination reaction rates as well as enhancing selectivity and reactivity. For instance, progress has been made in nucleophilic radiofluorination using Pd (Lee, E., Science 334:639 (2011); Kamlet, A. S., PLoS One 8:e59187 (2013)) and Ni (Lee, E., J. Am. Chem. Soc. 134:17456 (2012)) complexes. However, the requirement for the multistep synthesis of organometallic reagents under inert atmospheres has thus far limited adoption of these methods by non-experts.

Fluorination reactions disclosed in WO 2010/048170 also suffer from various deficiencies.

Additionally, methods of preparing radiofluorinating reagents often are not readily accessible to members of the radiochemistry community. Methods of preparing ¹⁸F silver fluoride, for example, involve specialized equipment (e.g., platinum reaction vessels and custom cyclotron targets) and/or insoluble silver sources (e.g., AgO, Ag₂CO₃, and silver wool) that are not readily adaptable to modern automated radiosynthesis modules.

### SUMMARY

The present invention is directed to methods of preparing Ag¹⁸F, to methods of radiofluorination using Ag¹⁸F, to compounds obtained by radiofluorination using Ag¹⁸F, and to diagnostic and therapeutic methods involving such compounds.

In one aspect, the present invention provides a method of preparing a radiolabeled aryl fluoride by reacting an aryl iodide having a formula ArI with Ag¹⁸F in the presence of a copper source. The reacting is carried out under conditions sufficient to form a radiolabeled aryl fluoride of Formula (1)

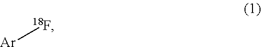

wherein Ar is an aryl group.

In another aspect, the invention provides a method of diagnosing or treating a disease or condition comprising administering a radiolabeled aryl fluoride as described herein to a subject in need thereof.

In another aspect, the invention provides a reaction mixture for radiofluorinating an aryl iodide precursor compound, the reaction mixture comprising an aryl iodide precursor compound having a formula ArI, Ag¹⁸F, and a copper source.

In another aspect, the invention provides a method of preparing Ag¹⁸F comprising eluting an ion exchange column with a water-soluble silver salt to obtain Ag¹⁸F, wherein the ion exchange column has been loaded with ¹⁸F.

In another aspect, the invention provides a kit comprising a water-soluble silver salt and an ion exchange column loaded with ¹⁸F.

In another aspect, the invention provides a method of preparing Ag¹⁸F comprising eluting an ion exchange column with a water-soluble salt to obtain a ¹⁸F salt, wherein the ion exchange column has been loaded with ¹⁸F, and combining the ¹⁸F salt with a silver salt to obtain Ag¹⁸F.

These and other embodiments and features of the present invention will become apparent from the following detailed description of the preferred embodiments.

### DETAILED DESCRIPTION

Disclosed herein are methods for radiolabeling aryl fluorides. The methods comprise reacting an aryl iodide with Ag¹⁸F in the presence of a copper source. The reacting is carried out under conditions sufficient to convert the aryl iodide to an aryl fluoride to provide the radiolabeled aryl fluoride. The disclosed methods provide radiolabeled compounds having a high specific activity and utilize starting materials including electron rich, electron neutral, and electron deficient arene substrates. The disclosed methods provide various radiolabeled compounds including, but not limited to, clinically relevant compounds such as 4-[¹⁸F]fluoro-m-hydroxyphenethylguanidine ([¹⁸F]MHPG; cardiac regional nerve density), 6-[¹⁸F]fluoro-L-DOPA (dopaminergic pathway), 3-[¹⁸F]fluoro-5-[(pyridine-2-yl)ethynyl] benzonitrile ([¹⁸F]FPEB; metabotropic glutamate receptor subtype 5), [¹⁸F]2′-methoxyphenyl-(N-2′-pyridinyl)-p-fluoro-benzamidoethylpiperazine ([¹⁸F]MPPF; 5-HT_{1A} receptor), [¹⁸F]octreotide (somatostatin 2 receptors in, e.g., neuroendocrine tumors), and [¹⁸F]Lipitor (HMG-CoA reductase inhibitor).

The use of the terms “a,” “an,” “the,” and similar referents in the context of describing the invention (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the invention and is not a limitation on the scope of the invention unless otherwise claimed. No language in the specification should be construed as indicating any non-claimed element as essential to the practice of the invention.

As used herein, the term “alkyl” refers to straight chained and branched hydrocarbon groups, including but not limited to, methyl, ethyl, n-propyl, i-propyl, n-butyl, sec-butyl, t-butyl, n-pentyl, 2-methylbutyl, 3-methylbutyl, 2,2-dimethylpropyl, n-hexyl, 2-methylpentyl, 3-methylpentyl, 4-methylpentyl, 2,2-dimethylbutyl, 2,3-dimethylbutyl, 3,3-dimethylbutyl, and 2-ethybutyl. The term Cₘ₋ₙ means the alkyl group has “m” to “n” carbon atoms. The term “alkylene” refers to an alkyl group having a substituent. An alkyl, e.g., methyl, or alkylene, e.g., —CH₂—, group can be substituted with one or more, and typically one to three, of independently selected halo, trifluoromethyl, trifluoromethoxy, hydroxy, alkoxy, nitro, cyano, alkylamino, or amino groups, for example.

As used herein, the term “halo” refers to fluoro, chloro, bromo, and iodo.

The term “hydroxy” is defined as —OH.

The term “alkoxy” is defined as —OR, wherein R is alkyl.

The term “amino” is defined as —NH₂, and the term “alkylamino” is defined as —NR₂, wherein at least one R is alkyl and the second R is alkyl or hydrogen.

The term “carbamoyl” is defined as —C(═O)NR₂.

The term “carboxy” is defined as —C(═O)OH or a salt thereof.

The term “nitro” is defined as —NO₂.

The term “cyano” is defined as —CN.

The term “trifluoromethyl” is defined as —CF₃.

The term “trifluoromethoxy” is defined as —OCF₃.

As used herein, the term “aryl” refers to a monocyclic or polycyclic aromatic group, preferably a monocyclic or bicyclic aromatic group. Examples of aryl groups include, but are not limited to, phenyl, naphthyl, fluorenyl, azulenyl, anthryl, phenanthryl, pyrenyl, biphenyl, and terphenyl. Aryl also refers to bicyclic and tricyclic carbon rings, where one ring is aromatic and the others are saturated, partially unsaturated, or aromatic, for example, dihydronaphthyl, indenyl, indanyl, or tetrahydronaphthyl (tetralinyl). Unless otherwise indicated, an aryl group can be unsubstituted or substituted with one or more, and in particular one to four, groups independently selected from, for example, halo, alkyl, alkenyl, —OCF₃, —NO₂, —CN, —NC, —OH, alkoxy, amino, alkylamino, —CO₂H, —CO₂alkyl, —OCOalkyl, aryl, and heteroaryl.

As used herein, the term “benzyl” refers to —CH₂-phenyl. Unless otherwise indicated, a benzyl group can be unsubstituted or substituted with one or more, and in particular one to four, groups independently selected from, for example, halo, alkyl, alkenyl, —OCF₃, —NO₂, —CN, —NC, —OH, alkoxy, amino, alkylamino, —CO₂H, —CO₂alkyl, —OCOalkyl, aryl, and heteroaryl.

As used herein, the term “heterocyclic” refers to a heteroaryl and heterocycloalkyl ring systems.

As used herein, the term “heteroaryl” refers to a monocyclic or bicyclic ring system containing one or two aromatic rings and containing at least one nitrogen, oxygen, or sulfur atom in an aromatic ring. Each ring of a heteroaryl group can contain one or two O atoms, one or two S atoms, and/or one to four N atoms, provided that the total number of heteroatoms in each ring is four or less and each ring contains at least one carbon atom. In certain embodiments, the heteroaryl group has from 5 to 20, from 5 to 15, or from 5 to 10 ring atoms. Examples of monocyclic heteroaryl groups include, but are not limited to, furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzothiophenyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, isothiazolyl, naphthyridinyl, oxazolopyridinyl, phthalazinyl, pteridinyl, purinyl, pyridopyridyl, pyrrolopyridyl, quinolinyl, quinoxalinyl, quiazolinyl, thiadiazolopyrimidyl, and thienopyridyl. Unless otherwise indicated, a heteroaryl group can be unsubstituted or substituted with one or more, and in particular one to four, substituents selected from, for example, halo, alkyl, alkenyl, —OCF₃, —NO₂, —CN, —NC, —OH, alkoxy, amino, alkylamino, —CO₂H, —CO₂alkyl, —OCOalkyl, aryl, and heteroaryl.

As used herein, the term “cycloalkyl” means a monocyclic or bicyclic, saturated or partially unsaturated, ring system containing three to eight carbon atoms, including cyclopropyl, cyclobutyl, cyclopentyl, cyclohexyl, cycloheptyl, and cyclooctyl, optionally substituted with one or more, and typically one to three, of independently selected halo, trifluoromethyl, trifluoromethoxy, hydroxy, alkoxy, nitro, cyano, alkylamino, or amino groups, for example.

As used herein, the term “heterocycloalkyl” means a monocyclic or a bicyclic, saturated or partially unsaturated, ring system containing 4 to 12 total atoms, of which one to five of the atoms are independently selected from nitrogen, oxygen, and sulfur and the remaining atoms are carbon. Nonlimiting examples of heterocycloalkyl groups are azetidinyl, pyrrolidinyl, piperidinyl, piperazinyl, dihydropyrrolyl, morpholinyl, thiomorpholinyl, dihydropyridinyl, oxacycloheptyl, dioxacycloheptyl, thiacycloheptyl, diazacycloheptyl, each optionally substituted with one or more, and typically one to three, of independently selected halo, C₁₋₆ alkyl, C₁₋₆ alkoxy, cyano, amino, carbamoyl, nitro, carboxy, C₂₋₇ alkenyl, C₂₋₇ alkynyl, or the like on an atom of the ring.

In one aspect, a method is provided for preparing a radiolabeled aryl fluoride comprising reacting an aryl iodide having a formula ArI with Ag¹⁸F in the presence of a copper source under conditions sufficient to form a radiolabeled aryl fluoride of Formula (1):

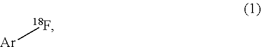

wherein Ar is an aryl group.

In another aspect, a reaction mixture is provided for radiofluorinating an aryl iodide precursor compound, the reaction mixture comprising an aryl iodide precursor compound having a formula ArI, Ag¹⁸F, and a copper source.

In some embodiments, Ar has a structure of Formula (2):

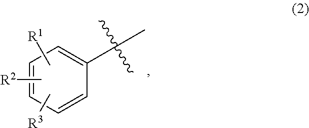

wherein R¹, R², and R³ are independently selected from the group consisting of H, C₁₋₄alkyl, OR^{a}, NR^{a}R^{b}, halo, —NR^{a}C(═O)R^{b}, —C(═O)NR^{a}R^{b}, —OC(═O)R^{a}, —C(═O)OR^{a}, —C(═O)R^{a}, aryl, benzyl, and

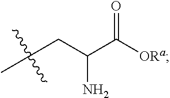

or
R² and R³, taken together with the carbon atoms to which they are attached, form a 4- to 8-membered ring; R^{a} is selected from the group consisting of H, C₁₋₄alkyl, aryl, and benzyl; and R^{b} is selected from the group consisting of H, C₁₋₄alkyl, aryl, benzyl, —O—C₁₋₄alkyl, —O-aryl, and —O-benzyl; with the proviso that at least one of R¹, R², and R³ is other than H.

Ar groups include, but are not limited to, the following:

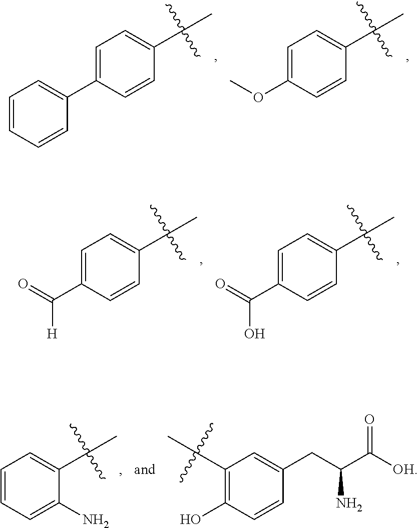

Compounds of formula ArI include, but are not limited to, the following:

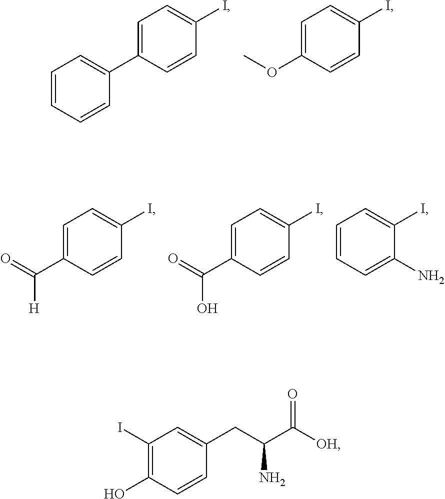

4-iodo-m-hydroxyphenethylguanidine, 6-iodo-L-DOPA, 3-iodo-5-[(pyridine-2-yl)ethynyl]benzonitrile, 2′-methoxyphenyl-(N-2′-pyridinyl)-p-iodo-benzamidoethylpiperazine, iodo-octreotide, and iodo-LIPITOR (LIPITOR is also known as atorvastatin).

Compounds of Formula (1) include, but are not limited to, the following:

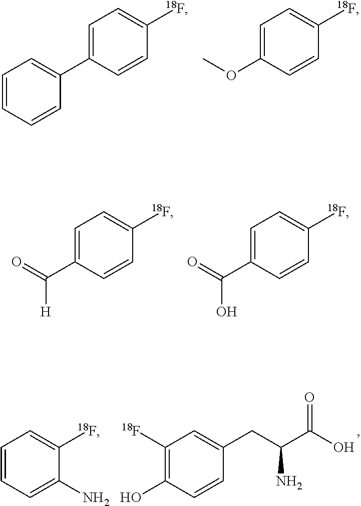

4-[¹⁸F]fluoro-m-hydroxyphenethylguanidine, 6-[¹⁸F]fluoro-L-DOPA, 3-[¹⁸F]fluoro-5-[(pyridine-2-yl)ethynyl]benzonitrile, [¹⁸F]2′-methoxyphenyl-(N-2′-pyridinyl)-p-fluoro-benzamidoethylpiperazine, [¹⁸F]octreotide, and [¹⁸F]LIPITOR (LIPITOR is also known as atorvastatin).

In various embodiments, the copper source includes, but is not limited to, copper(II) trifluoromethanesulfonate (Cu(OTf)₂), copper(II) carbonate basic (CuCO₃.Cu(OH)₂), copper(I) trifluoromethanesulfonate toluene complex (CuOTf.toluene), tetrakisacetonitrile copper(I) triflate ((CH₃CN)₄CuOTf), copper(I) trifluoromethanesulfonate bis(trimethylacetonitrile) complex ((tBuCN)₂CuOTf), ammonium tetrachlorocuprate(II), copper benzene-1,3,5-tricarboxylate, bis(1,3-bis(2,6-diisopropylphenyl)imidazol-2-ylidene)copper(I) tetrafluoroborate, bis[1,3-bis(2,4,6-trimethylphenyl)imidazol-2-ylidene]copper(I) tetrafluoroborate, bis(ethylenediamine)copper(II) hydroxide, (R,R)—(−)—N,N′-bis(3-hydroxylsalicylidene)-1,2-cyclohexanediaminocopper(II)samarium isopropoxide, bis[(tetrabutylammonium iodide)copper(I) iodide], [bis(trimethylsilyl)acetylene](hexafluoroacetylacetonato)copper(I), bromotris(triphenylphosphine)copper(I), 5-chlorobenzo[b]phosphindole, chloro[1,3-bis(2,6-diisopropylphenyl)imidazol-2-ylidene]copper(I), copper(I) acetate, copper(II) acetate 1,2-bis(diphenylphosphino)ethane, copper(II) acetylacetonate, copper(I) bromide, copper(I) bromide dimethyl sulfide complex, copper(II) tert-butylacetoacetate, copper(II) carbonate, copper(I) chloride, copper(II) chloride, copper(I) chloride-bis(lithium chloride) complex, copper(I) cyanide di(lithium chloride) complex, copper(II) 3,5-diisopropylsalicylate, copper (I) diphenylphosphinate, copper(II) ethylacetoacetate, copper(II) 2-ethylhexanoate, copper formate, copper hydride, copper(I) iodide, copper iodide dimethyl sulfide complex, copper(I) iodide trimethylphosphite complex, copper(I) 3-methylsalicylate, copper(II) nitrate, copper(I) oxide, copper oxychloride, copper(II) sulfate, copper(II) tartrate, copper(II) tetrafluoroborate, copper(I) thiophene-2-carboxylate, copper(I) thiophenolate, di-p-hydroxo-bis[(N,N,N′,N′-tetramethylethylenediamine)copper(II)] chloride, copper(I) trifluoromethanesulfonate benzene complex, cupric carbonate, {Cuprous 2-[(2-diphenylphosphino)benzylideneamino]-3,3-dimethylbutyrate,triflatesodium triflate}complex, (1,4-diazabicyclo[2.2.2]octane)copper(I) chloride complex, dichloro(1,10-phenanthroline)copper(II), dilithium tetrachlorocuprate(II), hydro[(4R)-[4,4′-bi-1,3-benzodioxole]-5,5′-diylbis[bis[3,5-bis(1,1-dimethylethyl)-4-methoxyphenyl]phosphine-P]]copper(I), (ethylcyclopentadienyl)(triphenylphosphine)copper(I), fluorotris(triphenylphosphine)copper(I), iodo(triethyl phosphite)copper(I), mesitylcopper(I), (1,10-phenanthroline)bis(triphenylphosphine)copper(I) nitrate dichloromethane adduct, phthalocyanine green, tetrakis(acetonitrile)copper(I) hexafluorophosphate, tetrakis(acetonitrile)copper(I) tetrafluoroborate, and tetrakis(pyridine)copper(II) triflate.

In various embodiments, the reaction mixture further comprises a crown ether such that the reacting is carried out in the presence of the crown ether. Crown ethers include, but are not limited to, 12-crown-4, 15-crown-5, 18-crown-6, dibenzo-18-crown-6, and diaza-18-crown-6.

The ¹⁸F fluorination reaction can be carried out in various solvents. Suitable solvents include, but are not limited to, polar protic solvents, polar aprotic solvents, nonpolar solvents, alcohols, esters, ethers, amides, glycols, glycol ethers, aliphatic and aromatic hydrocarbons, chlorinated solvents, C₁₋₆alcohols (e.g., methanol, ethanol, propyl alcohol, and butyl alcohol, including isomers thereof), monoC₁₋₄alkyl ethers of ethylene glycol and propylene glycol, acetone, methyl ethyl ketone, isophorone, dichloromethane, chloroform, ethyl acetate, 2-methoxyethanol, dimethylformamide (DMF), dimethyl sulfoxide (DMSO), tetrahydrofuran (THF), acetonitrile, trimethylacetonitrile (also known as tert-butyl cyanide, tBuCN, pivalonitrile), kerosene, mineral spirits, xylene, toluene, and mixtures thereof such as a mixture of tBuCN and acetonitrile. Suitable volume ratios of tBuCN to acetonitrile include, but are not limited to, about 1:1 to about 5:1, about 1.5:1 to about 4.5:1, about 2:1 to about 4:1, about 2.5:1 to about 3.5:1, and/or about 3:1.

The ¹⁸F fluorination reaction can be carried out at various molar ratios copper source to aryl iodide. Suitable molar ratios of copper source to aryl iodide include, but are not limited to, about 1:10 to about 10:1, about 1:5 to about 5:1, about 1:2 to 3:1, and/or about 1:1 to about 3:1.

The ¹⁸F fluorination reaction can be carried out at various molar ratios aryl iodide to Ag¹⁸F. Suitable molar ratios of aryl iodide to Ag¹⁸F include, but are not limited to, about 10000000:1 to about 1000:1, about 5000000:1 to about 10000:1, about 1000000:1 to about 100000:1.

The ¹⁸F fluorination reaction can be carried out at various loading levels of the aryl iodide. Suitable loading levels of the aryl iodide include, but are not limited to, about 1 μmol or greater, about 2 μmol or greater, about 3 μmol or greater, about 4 μmol or greater, about 5 μmol or greater, about 6 μmol or greater, about 10 μmol or greater, about 20 μmol or greater, about 30 μmol or greater, about 50 μmol or greater, about 1 μmol to about 100 μmol, about 2 μmol to about 50 μmol, about 3 μmol to about 30 μmol, and/or about 5 μmol to about 20 μmol.

The ¹⁸F fluorination reaction can be carried out at various temperatures. Suitable reaction temperatures include, but are not limited to, a temperature of about 20° C. to about 200° C., such as about 50° C. to about 190° C., about 80° C. to about 180° C., about 100° C. to about 160° C., and/or about 120° C. to about 150° C.

The ¹⁸F fluorination reaction can be carried out for various lengths of time. Suitable reaction times include, but are not limited to, a reaction time of about 1 minute to about 180 minutes, about 5 minutes to about 120 minutes, about 10 minutes to about 100 minutes, about 15 minutes to about 90 minutes, about 20 minutes to about 80 minutes, about 30 minutes to about 70 minutes, and/or about 45 minutes to about 60 minutes.

¹⁸F-labeled aryl fluorides of Formula (1) prepared according to the methods disclosed herein are obtained in high radiochemical yield (RCY). For example, the products of the methods disclosed herein are obtained in a radiochemical yield of about 2% or greater, about 5% or greater, about 10% or greater, about 20% or greater, about 30% or greater, about 40% or greater, about 50% or greater, about 60% or greater, and/or about 70% or greater.

In various embodiments, the radiolabeled aryl fluoride of Formula (1) is isolated. Suitable methods of isolating the radiolabeled aryl fluoride of Formula (1) include, but are not limited to, extraction, chromatography, and crystallization.

In one aspect, the disclosure provides a method of diagnosing or treating a disease or condition comprising administering a radiolabeled aryl fluoride as described herein to a subject in need thereof.

In another aspect, the disclosure provides a method of preparing Ag¹⁸F comprising eluting an ion exchange column with a water-soluble silver salt to obtain Ag¹⁸F, wherein the ion exchange column has been loaded with ¹⁸F⁻.

In another aspect, the disclosure provides a kit comprising a water-soluble silver salt and an ion exchange column loaded with ¹⁸F⁻.

In various embodiments, the ion exchange column, prior to loading with ¹⁸F⁻, has been equilibrated with an aqueous solution comprising a triflate (⁻OTf) salt, an acetate salt, a tosylate (⁻OTs) salt, a mesylate (⁻OMs) salt, or a nitrate (⁻NO₃) salt.

In various embodiments, the ion exchange column is a quaternary ammonium column, such as a quaternary methyl ammonium column. Exemplary ion exchange columns include, but are not limited to, Sep-Pak cartridges, such as quaternary methyl ammonium (QMA) ion exchange Sep-Pak cartridges.

In various embodiments, the water-soluble silver salt is selected from the group consisting of AgBF₄, silver triflate (AgOTf), silver acetate (AgOAc), silver tosylate (AgOTs), silver mesylate (AgOMs), silver nitrate (AgNO₃), silver trifluoroacetate (AgTFA), silver perchlorate (AgClO₄), and tetrakis(acetonitrile)silver(I) tetrafluoroborate ([(CH₃CN)₄Ag]BF₄). Water-soluble silver salts typically have a solubility in water of at least about 0.1 mg/mL, at least about 0.5 mg/mL, at least about 1 mg/mL, at least about 2 mg/mL, at least about 3 mg/mL, at least about 5 mg/mL, at least about 10 mg/mL, at least about 20 mg/mL, at least about 50 mg/mL, and/or at least about 100 mg/mL.

In another aspect, the disclosure provides a method of preparing Ag¹⁸F comprising eluting an ion exchange column with a water-soluble salt (e.g., KOTf or KOAc) to obtain a ¹⁸F salt (e.g., a K¹⁸F salt), wherein the ion exchange column has been loaded with ¹⁸F⁻, and combining the ¹⁸F salt with a silver salt (e.g., AgOTf) to obtain Ag¹⁸F. In various embodiments, the ion exchange column, prior to loading with ¹⁸F⁻, has been equilibrated with an aqueous solution comprising a water-soluble salt (e.g., KOTf or KOAc).

### EXAMPLES

### Example 1

### Synthesis of Ag¹⁸F Via Elution with Water-Soluble Silver Salts

A solution of ¹⁸F⁻ in [¹⁸O]H₂O was cyclotron produced via the ¹⁸O(p,n)¹⁸F nuclear reaction. A quaternary methyl ammonium (QMA) ion exchange Sep-Pak cartridge was conditioned with potassium triflate (KOTf) or potassium acetate (KOAc). The solution of ¹⁸F⁻ in H₂¹⁸O was passed through the conditioned Sep-Pak cartridge to trap ¹⁸F⁻ on the cartridge. The cartridge was then eluted with an aqueous solution of a silver salt to generate Ag¹⁸F. Elution with an aqueous solution of AgO or Ag₂CO₃ was not effective in eluting Ag¹⁸F from the cartridge. Without wishing to be bound by theory, it is believed that AgO and Ag₂CO₃, which are sparingly soluble in water, are ineffective in eluting Ag¹⁸F from the cartridge because they clog the frits, lines, and sep-paks used in radiosynthesis. When aqueous solutions of more water-soluble silver salts, including AgBF₄, silver triflate (AgOTf), silver acetate (AgOAc), AgNO₃, and tetrakis(acetonitrile)silver(I) tetrafluoroborate ([(CH₃CN)₄Ag]BF₄), were used to elute the cartridge, Ag¹⁸F was obtained in high yields (88-98%).

### Example 2

### Synthesis of ¹⁸F-Labeled Biphenyl

4-[¹⁸F]Biphenyl was prepared according to the following procedure. To an aqueous solution of Ag¹⁸F was added 18-crown-6 in acetonitrile and the mixture was azeotropically dried. The dried mixture was reconstituted in DMF and added to a mixture of copper(I) trifluoromethanesulfonate toluene complex (CuOTf.toluene) and 4-iodobiphenyl (0.06 mmol) in DMF (3:1 ratio of copper catalyst:4-iodobiphenyl; ˜10⁶-fold excess relative to Ag¹⁸F). The reaction mixture was heated to 140° C. for 40 min. The product 4-[¹⁸F]biphenyl was obtained from 4-iodobiphenyl with 4% radiochemical conversion (RCC).

### Example 3

### Synthesis of ¹⁸F-Labeled Tyrosine

3-[¹⁸F]Tyrosine was prepared according to the following procedure. An aqueous solution of Ag¹⁸F and acetonitrile was azeotropically dried. The dried mixture was reconstituted in acetonitrile and added to a mixture of copper(I) trifluoromethanesulfonate bis(trimethylacetonitrile) complex ((tBuCN)₂CuOTf) (0.015 mmol) and 3-iodotyrosine (0.01 mmol) in trimethylacetonitrile (0.05 M) that had been stirred overnight to give a ratio of 3:1 for trimethylacetonitrile to acetonitrile (3:1 ratio of copper catalyst:3-iodotyrosine; ˜10⁶-fold excess relative to Ag¹⁸F). The reaction mixture was heated to 140° C. for 60 min. The product 3-[¹⁸F]fluorotyrosine was obtained from 3-iodotyrosine with a 47% radiochemical conversion (RCC).

### Example 4

### Synthesis of Ag¹⁸F Via Elution with Water-Soluble Silver Salts

A quaternary methyl ammonium (QMA) ion exchange Sep-Pak cartridge was conditioned with potassium triflate (KOTf) or potassium acetate (KOAc). [¹⁸F]Fluoride was produced via the ¹⁸O(p,n)¹⁸F nuclear reaction (using a GE PETTrace cyclotron at 40 μA beam for 2 min, which generated ca. 150 mCi of [¹⁸F]fluoride). The solution of ¹⁸F⁻ in H₂¹⁸O was passed through the conditioned Sep-Pak cartridge to trap ¹⁸F⁻ on the cartridge. The cartridge was then eluted with an aqueous solution of a KOTf or KOAc (5 mg) salt to generate K¹⁸F that was obtained in high yields (88-98%). The K¹⁸F was added to AgOTf to generate soluble Ag¹⁸F to use in reactions.

The present invention is described in connection with preferred embodiments. However, it should be appreciated that the invention is not limited to the disclosed embodiments. It is understood that, given the description of the embodiments of the invention herein, various modifications can be made by a person skilled in the art. Such modifications are encompassed by the claims below.

What is claimed is:

1. A method of preparing a radiolabeled aryl fluoride comprising:
reacting an aryl iodide having a formula ArI with Ag¹⁸F in the presence of a copper source under conditions sufficient to form a radiolabeled aryl fluoride of Formula (1) 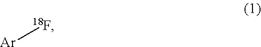
wherein Ar is an aryl group.

2. The method of claim 1, wherein Ar has a structure of Formula (2): 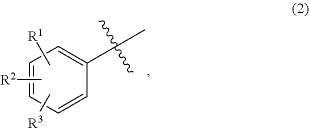
wherein R¹, R², and R³ are independently selected from the group consisting of H, C₁₋₄alkyl, OR^{a}, NR^{a}R^{b}, halo, —NR^{a}C(═O)R^{b}, —C(═O)NR^{a}R^{b}, —OC(═O)R^{a}, —C(═O)OR^{a}, —C(═O)R^{a}, aryl, benzyl, and 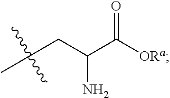
or
R² and R³, taken together with the carbon atoms to which they are attached, form a 4- to 8-membered ring;
R^{a} is selected from the group consisting of H, C₁₋₄alkyl, aryl, and benzyl; and
R^{b} is selected from the group consisting of H, C₁₋₄alkyl, aryl, benzyl, —O—C₁₋₄alkyl, —O-aryl, and —O-benzyl;
with the proviso that at least one of R¹, R², and R³ is other than H.

3. The method of claim 1, wherein Ar is selected from the group consisting of:

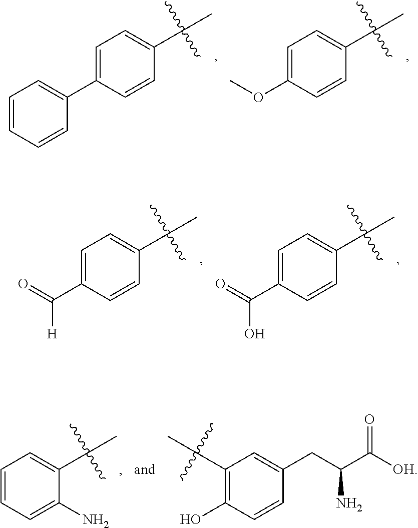

4. The method of claim 1, wherein Formula (1) is selected from the group consisting of 4-[¹⁸F]fluoro-m-hydroxyphenethylguanidine, 6-[¹⁸F]fluoro-L-DOPA, 3-[¹⁸F]fluoro-5-[(pyridine-2-yl)ethynyl] benzonitrile, [¹⁸F]2′-methoxyphenyl-(N-2′-pyridinyl)-p-fluoro-benzamidoethylpiperazine, [¹⁸F]octreotide, and [¹⁸F]Lipitor.

5. The method of claim 1, wherein the copper source is selected from the group consisting of Cu(OTf)₂, CuCO₃.Cu(OH)₂, CuOTf.toluene, (CH₃CN)₄CuOTf, and (tBuCN)₂CuOTf.

6. The method of claim 1, wherein the reaction mixture further comprises a crown ether.

7. The method of claim 6, wherein the crown ether is selected from the group consisting of 12-crown-4, 15-crown-5, 18-crown-6, dibenzo-18-crown-6, and diaza-18-crown-6.

8. The method of claim 1, wherein the reacting is carried out at a temperature of about 20° C. to about 200° C., such as about 50° C. to about 190° C., about 80° C. to about 180° C., about 100° C. to about 160° C., and/or about 120° C. to about 150° C.

9. The method of claim 1, wherein the reacting is carried out at a molar ratio of the copper source to the aryl iodide of about 1:10 to about 10:1, about 1:5 to about 5:1, about 1:2 to 3:1, and/or about 1:1 to about 3:1.

10. The method of claim 1, wherein the reacting is carried out in a polar aprotic solvent.

11. The method of claim 1, wherein the reacting is carried out in a solvent selected from the group consisting of acetone, methyl ethyl ketone, isophorone, dichloromethane, chloroform, ethyl acetate, dimethylformamide (DMF), dimethyl sulfoxide (DMSO), tetrahydrofuran (THF), acetonitrile, trimethylacetonitrile (tBuCN), and mixtures thereof such as a mixture of tBuCN and acetonitrile.

12. The method of claim 1, further comprising isolating the radiolabeled aryl fluoride of Formula (1).

13. A method of diagnosing or treating a disease or condition comprising administering the radiolabeled aryl fluoride of claim 1 to a subject in need thereof.

14. A reaction mixture for radiofluorinating an aryl iodide precursor compound, the reaction mixture comprising an aryl iodide precursor compound having a formula ArI, Ag¹⁸F, and a copper source.

15. The reaction mixture of claim 14, wherein Ar has a structure of Formula (2) as defined in claim 2.

16. The reaction mixture of claim 14, wherein Ar is selected from the group consisting of:

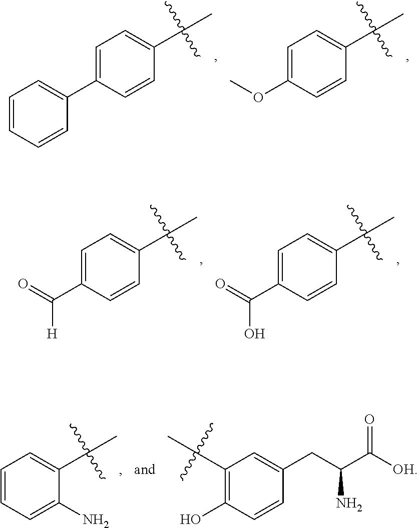

17. The reaction mixture of claim 14, wherein the copper source is selected from the group consisting of Cu(OTf)₂, CuCO₃.Cu(OH)₂, CuOTf.toluene, and (CH₃CN)₄CuOTf.

18. The reaction mixture of claim 14, wherein the reaction mixture further comprises a crown ether.

19. The reaction mixture of claim 18, wherein the crown ether is selected from the group consisting of 12-crown-4, 15-crown-5, 18-crown-6, dibenzo-18-crown-6, and diaza-18-crown-6.

20. The reaction mixture of claim 14, wherein the molar ratio of the copper source to the aryl iodide is about 1:10 to about 10:1, about 1:5 to about 5:1, about 1:2 to 3:1, and/or about 1:1 to about 3:1.

21. The reaction mixture of claim 14, further comprising a polar aprotic solvent.

22. The reaction mixture of claim 21, wherein the polar aprotic solvent is selected from the group consisting of acetone, methyl ethyl ketone, isophorone, dichloromethane, chloroform, ethyl acetate, dimethylformamide (DMF), dimethyl sulfoxide (DMSO), tetrahydrofuran (THF), acetonitrile, trimethylacetonitrile (tBuCN), and mixtures thereof such as a mixture of tBuCN and acetonitrile.

23. A method of preparing Ag¹⁸F comprising eluting an ion exchange column with a water-soluble silver salt to obtain Ag¹⁸F, wherein the ion exchange column has been loaded with ¹⁸F⁻.

24. The method of claim 23, wherein the ion exchange column, prior to loading with ¹⁸F⁻, has been equilibrated with an aqueous solution comprising a triflate (⁻OTf) salt, an acetate salt, a tosylate (⁻OTs) salt, a mesylate (⁻OMs) salt, or a nitrate (⁻NO₃) salt.

25. The method of claim 23, wherein the ion exchange column is a quaternary ammonium column.

26. The method of claim 25, wherein the quaternary ammonium column is a quaternary methyl ammonium column.

27. The method of claim 23, wherein the water-soluble silver salt is selected from the group consisting of AgBF₄, silver triflate (AgOTf), silver acetate (AgOAc), silver tosylate (AgOTs), silver mesylate (AgOMs), silver nitrate (AgNO₃), silver trifluoroacetate (AgTFA), silver perchlorate (AgClO₄), and tetrakis(acetonitrile)silver(I) tetrafluoroborate ([(CH₃CN)₄Ag]BF₄).

28. A kit comprising a water-soluble silver salt and an ion exchange column loaded with ¹⁸F⁻.

29. The kit of claim 28, wherein the ion exchange column is a quaternary ammonium column.

30. The kit of claim 28, wherein the water-soluble silver salt is selected from the group consisting of AgBF₄, silver triflate (AgOTf), silver acetate (AgOAc), silver tosylate (AgOTs), silver mesylate (AgOMs), silver nitrate (AgNO₃), silver trifluoroacetate (AgTFA), silver perchlorate (AgClO₄), and tetrakis(acetonitrile)silver(I) tetrafluoroborate ([(CH₃CN)₄Ag]BF₄).

31. A method of preparing Ag¹⁸F comprising eluting an ion exchange column with a water-soluble salt to obtain a ¹⁸F salt, wherein the ion exchange column has been loaded with ¹⁸F⁻, and combining the ¹⁸F salt with a silver salt to obtain Ag¹⁸F.

32. The method of claim 31, wherein the ion exchange column, prior to loading with ¹⁸F⁻, has been equilibrated with an aqueous solution comprising a water-soluble salt.

33. The method of claim 31, wherein the ion exchange column is a quaternary ammonium column.

34. The method of claim 33, wherein the quaternary ammonium column is a quaternary methyl ammonium column.

35. The method of claim 31, wherein the water-soluble salt is KOTf or KOAc.

36. The method of claim 31, wherein the silver salt is selected from the group consisting of AgBF₄, silver triflate (AgOTf), silver acetate (AgOAc), silver tosylate (AgOTs), silver mesylate (AgOMs), silver nitrate (AgNO₃), silver trifluoroacetate (AgTFA), silver perchlorate (AgClO₄), and tetrakis(acetonitrile)silver(I) tetrafluoroborate ([(CH₃CN)₄Ag]BF₄).
